(19) United States
(12) Patent Application Publication
(10) Pub. No.: US 2007/0259409 A1
(43) Pub. Date: Nov. 8, 2007

(54) Microbial Production of Aromatic Acids

(75) Inventor: Jan Wery, Gorssel (NL)

Correspondence Address: WEINGARTEN, SCHURGIN, GAGNEBIN & LEBOVICI LLP, TEN POST OFFICE SQUARE, BOSTON, MA, 02109, US

(73) Assignee: NEDERLANDSE ORGANISATIE VOOR TOEGEPASTNATUURWETENS, Delft (NL)

(21) Appl. No.: 11/578,824

(22) PCT Filed: Dec. 23, 2004
(86) PCT No.: PCT/NL04/00905

§ 371 Date: Jul. 3, 2007

(30) Foreign Application Priority Data
Apr. 21, 2004 (EP) 04076209.8

(51) Int. Cl. C12P 7/40; C12N 1/00; C12P 7/42; C12N 15/01

(52) U.S. Cl. 435/146; 435/136; 435/243; 435/440

(57) ABSTRACT

The invention relates to the enzymatic production of aromatic acids using renewable carbon sources, such as sugars. Provided is a method for the microbial production of aromatic acids from a fermentable carbon substrate using a host cell capable of producing said aromatic acid, for instance cinnamic acid, para-hydroxycinnamic acid and para-hydroxybenzoic acid, and comprising an efflux pump for said aromatic acid. A preferred host cell comprises a member of the proton-dependent resistance/nodulation/cell division (RND) family of efflux pumps, preferably the solvent resistance pump srpABC of P. putida strain S12.

The invention relates to the production of aromatic acids from renewable carbon sources, such as sugars. More specifically, it relates to an improved method for the microbial production of aromatic acids using a host cell comprising an enzymatic pump capable of actively secreting said aromatic acid out of said host cell, for example into the culture medium. Whereas the invention will be mainly described with reference to the production of cinnamic acid, para-hydroxy cinnamic acid and para-hydroxybenzoic acid, a method of the invention is also advantageously used for the microbial production of other aromatic acids.

The chemical synthesis of aromatics is based on non-renewable carbon substrates, oftentimes demanding much energy and/or expensive chemical activating and protection groups and/or large amounts of solvents. Thus in many cases chemical synthesis of aromatics is undesirable from an environmental point of view. Furthermore, the chemical synthesis of aromatic acids is often laborious. Therefore, large scale synthesis of aromatic acids is preferably performed using a microbial production system and renewable carbon sources, such as sugars.

Examples of commercially important aromatic acids include cinnamic acid (referred to as CA), para-hydroxycinnamic acid (referred to as PHCA) and para-hydroxybenzoic acid (referred to as PHB). PHCA is a useful monomer for the production of Liquid Crystal Polymers (LCP). LCP's may be used in electronic connectors and telecommunication and aerospace applications. LCP resistance to sterilizing radiation has also enabled these materials to be used in medical devices as well as chemical, and food packaging applications. Furthermore, PHCA can be used in sun screen products and cosmetics and as antioxidant in food stuff. An important pharmaceutical for high blood pressure and stroke prevention, known as coumarin or oxy-cinnamic acid, is a derivative of CA. For an overview of the occurrence and metabolism of CA and related compounds see a Review by J. A. Hoskins (1984; Journal of Applied Toxicology, 4:283-292). PHB is also used as a monomer for synthesis of LCP's. It is also a food preservative and is used as a stabilizer in cosmetic preparations. Also, PHB is used as a chemical intermediate for synthetic drugs, pharmaceuticals, dyes and plasticizers. Esters of PHB are known as parabens, which are used as antimicrobial preservatives in deodorants, antiperspirants and in a wide range of other consumer products.

CA and PHCA both occur naturally in plants, where they serve as intermediates in the lignin biosynthetic pathway in plants (Plant Biochemistry, Ed. P. M. Dey, Academic Press, 1997). Methods of isolation and purification of CA and PHCA from plants are known (R. Benrief, et al., Phytochemistry, 1998, 47, 825-832; WO 97/2134).

Chemical synthesis of PHB has been described previously (JP 05009154; U.S. Pat. No. 5,399,178; U.S. Pat. No. 4,740,614; U.S. Pat. No. 3,985,797).

The methods of obtaining said aromatic acids are time consuming and cumbersome and/or energy demanding, and a biological method of production could offer a simplified and sustainable solution.

The microbial production of PHB was described in WO 01/92539. This patent differs from the present invention in that PHB was not produced from a renewable feedstock, such as sugar, but from the preformed substrate toluene. Unlike in the present invention, this process offers no solution with respect to sustainability.

The microbial production of CA and PHCA using a sugar fermentation route is known from WO 02/090523, wherein a microbial host cell is engineered with key enzymes involved in aromatic acid synthesis such that it produces the desired aromatic acid(s). However, further optimization and improvement of the product yield of known microbial production methods is hampered by the fact that aromatic acids, once produced, typically accumulate in the host cell. As a result, the accumulated product inhibits one or more enzymes involved in its production such that a further increase in aromatic acid synthesis is restrained. This phenomenon is also referred to as a negative feedback control mechanism.

The invention now provides the insight that a host cell comprising an efflux pump that is capable of actively transporting an aromatic acid out of the cell is advantageously used for the microbial production of aromatic acids.

Provided is a method for the microbial production of an aromatic acid from a renewable carbon substrate using a host cell capable of producing said aromatic acid and comprising an efflux pump for said aromatic acid. In a method of the invention, the host cell can secrete the aromatic acid into the culture medium, such that product accumulation in the cell and, conceivably, negative feedback, is minimized. As a result, higher product yields can be achieved compared to existing microbial production systems that employ host cells which cannot effectively secrete the aromatic acid produced. In addition, a method of the invention does not require the harvest and further processing of host cells to obtain the desired end product. Instead, the culture medium of the host cell enriched with the end product can be taken and subjected to further processing to isolate and/or purify the product. Thus, a method is provided simple in its use that can be performed in a continuous fashion, whereas a batch wise cultivation is possible as well.

A renewable carbon substrate refers to a carbon source of plant or animal origin. It can be used by the host cell as a growth substrate and is typically part of a fermentable culture medium of a host cell. Examples of renewable carbon substrates include carbohydrates, such as glucose, fructose, galactose, mannose, mannitol, sucrose, starch, starch hydrolyzate, and molasses; alcohols such as methyl alcohol, ethyl alcohol, propyl alcohol, butyl alcohol, and glycerine; and organic acids.

Suitable host cells for use in a method of the invention include microbial cells which can produce an aromatic acid and which display a tolerant phenotype towards hydrophobic solvents such as toluene and octanol. However, also (bacterial) host cells which are not solvent-tolerant but which do comprise an efflux pump capable of exporting aromatic acids are of use in the present invention.

Many different mechanisms have been described that contribute to solvent tolerance, one of which relates to an energy-dependent efflux pump which actively keeps toxic solvents out of the interior of the cell. Solvent tolerant host cells are advantageously used in a method of the invention because the pump conferring resistance or tolerance towards organic solvents has been shown to possess a very broad specificity, taking organic compounds that by virtue of their chemico-physical characteristics accumulate into the bacterial membrane such as aromatics, alcohols, alkanes etc. as a substrate (Kieboom et al. 1998. J. Biol. Chem. 273:85-91). Undissociated aromatic acids will by virtue of similar chemico-physical characteristics also partition effectively to the cell membrane where they act as a substrate of such a pump.

In one embodiment of the invention, a host cell, preferably a Gram-negative bacterium, comprises a member of the proton-dependent resistance/nodulation/cell division (RND) family of efflux pumps. RND-type efflux pumps belong to the multidrug resistance (MDR) pumps. They have an extremely broad substrate specificity and protect bacterial cells from the actions of antibiotics on both sides of the cytoplasmic membrane. Members of this family have been shown to be involved in export of antibiotics, metals, and oligosaccharides involved in nodulation signalling. RND-type efflux pumps usually function as three-component assemblies spanning the outer and cytoplasmic membranes and the periplasmic space of Gram-negative bacteria. Examples of suitable RND-type efflux pumps for use in a method of the invention can be found in Tseng, T. T., Gratwick, K. S., Kollman, J., Park., D., Nies, D. H., Goffeau, A., & Saier Jr., M. H. (1999), J. Mol. Microbiol. Biotechnol. 1: 107-125.

In one embodiment, a method provided herein for the production of an aromatic acid from a renewable carbon source uses a host cell comprising a solvent resistance pump, preferably the solvent resistance pump srpABC of P. putida S12 (Isken et al. 1996 J. Bacteriol. 178:6056; Kieboom et al. 1998. J. Biol. Chem. 273:85-91). The deduced amino acid sequences of the proteins encoded by the srpABC genes have extensive homology with those of the RND family of efflux pumps. It is composed of three protein components that together span the inner and outer membranes of Gram-negative bacteria: an inner membrane transporter (SrpB analogues), an outer membrane channel (SrpC analogues), and a periplasmic linker protein (SrpA analogues). Dendrograms showing the phylogenetic relationship of SrpA, SrpB, and SrpC to other proteins involved in multidrug resistance are shown in Kieboom et al. 1998 J. Biol. Chem. 273:85-91. The srpABC-encoded proteins show the most homology with those for the mexAB/oprM-encoded multidrug resistance pump found in Pseudomonas aeruginosa. SrpA, SrpB, and SrpC are 57.8, 64.4, and 58.5% identical to MexA, MexB, and OprM, respectively. In one embodiment of the present invention, a host cell comprises an efflux pump consisting of an inner membrane transporter, an outer membrane channel, and a periplasmic linker protein belonging to the RND-family of efflux pumps wherein the proteins show a homology of at least 50%, preferably at least 55% to the SrpA, SrpB or SrpC proteins of P. putida S12. In fact, any functional equivalent of known solvent efflux pumps that can use an aromatic acid as a substrate are suitably used in a method of the invention.

The article by Kieboom et al. relates to solvent-tolerance of bacteria. It discusses the role of proteins involved in proton-dependent efflux systems, such as those encoded by the srpABC genes of P. putida, in mediating resistance towards (exogenous) toxic molecules, for example toxic hydrocarbon substrates that are utilized by the bacteria as a carbon source or toxic nonpolar solvents (e.g. toluene) that are advantageously used in the microbial production of fine chemicals to present hydrophobic substrates to the bacteria. De Bont et al. (Trends in Biotechnology 1998, Vol. 16, p. 493-499) also relate to toxicity problems encountered with the microbial production of fine chemicals. Specifically, it describes the use of solvent-tolerant bacteria that allow the use of organic solvents to extract toxic products from the aqueous phase during fermentation. Ramos et al. (J. of Bacteriology Vol. 180, p. 3323) also relates to solvent-resistant bacteria. It describes toluene and octanol tolerance in the P. putida strain DOT-TIE and the generation and characterization of a toluene-sensitive octanol-tolerant mutant. The mutation was found in a gene homologous to the mexB gene, which belongs to the efflux pump family of the RND-type. Thus, the prior art does not disclose or suggest methods for the microbial production of aromatic acids from a renewable carbon source as disclosed in the present invention. They merely teach that efflux pumps are useful for the export of exogenously added, toxic molecules (substrate, substrate solvent or product solvent) that have been chemically synthesized from non-renewable (i.e. fossile) carbon sources. In contrast, the aromatic acids produced by a host cell of the invention to be exported by an efflux pump are not exogenously added toxic molecules. Rather, they typically are naturally occurring molecules produced by the host cell itself. The invention surprisingly shows that efflux pumps are also suitably used to export aromatic acids that are produced endogenously from a renewable carbon source. As is demonstrated in the examples, export of the aromatic acids significantly increases aromatic acid production.

In a preferred embodiment, a method of the invention uses a Pseudomonas spp., preferably Pseudomonas putida, more preferably P. putida strain S12 as a host cell for the production of an aromatic acid.

In one embodiment, a method is provided for the microbial production of CA, PHCA and PHB using a host cell that can convert the fermentable carbon substrate into an aromatic amino acid (phenylalanine or tyrosine), which is subsequently converted into the aromatic acids CA, PHCA and PHB. Once produced, these aromatic acids are actively transported out of the host cell by an efflux pump, preferably by a member of the proton-dependent resistance/nodulation/cell division (RND) family of efflux pumps, more preferably srpABC.

Phenylalanine and tyrosine, which in a method of the invention serve as precursors for aromatic acids, are naturally present in micro-organisms. However, for an optimal synthesis of aromatic acids a host cell preferably over-produces one or more aromatic acid precursors (e.g. aromatic amino acids) such that the substrate level does not limit aromatic acid production by the host cell. Methods to increase aromatic amino acid synthesis in a micro-organism are known in the art. In one embodiment, a host cell is selected for increased resistance against a toxic analogue of an aromatic amino acid. For example, mutant micro-organisms can be selected for resistance to toxic (m-fluoro-)analogues of phenylalanine or tyrosine. These insensitive mutants often produce high levels of phenylalanine and tyrosine (GB 1071935; U.S. Pat. No. 3,709,785).

It is also possible to obtain a recombinant host cell with increased phenylalanine and tyrosine production by overexpression of one or more key genes in the biosynthesis of phenylalanine and/or tyrosine (Ikeda 2003. Amino acid production processes. P. 1-35. in T. Scheper (Ed.), Advances in Biochemical Engineering/Biotechnology, Vol. 79. Springer-Verlag, Berlin Heidelberg).

A recombinant micro-organism with increased resistance to m-fluoro-phenylalanine is advantageously used as a host cell in a method for the microbial production of CA, PHCA and PHB. Phenylalanine is enzymatically converted into CA through the action of phenylalanine ammonia lyase (PAL; EC 4.3.1.5). Therefore, a host cell of the invention preferably comprises, in addition to an efflux pump, at least one gene encoding PAL activity. The term “PAL activity” refers to the ability of a protein to catalyze the conversion of phenylalanine to CA.

In plants, CA is subsequently converted to PHCA by cinnamate-4-hydroxylase (C4H), a cytochrome P450-dependent monooxygenase (P450). Thus, it is evident that one possible route to PHCA is via phenylalanine ammonia lyase (PAL) from phenylalanine. However this route also requires the presence of the second enzyme, cinnamate-4-hydroxylase, an enzyme which is rare in most micro-organisms. Information available indicates that PAL from some plants and micro-organisms can accept tyrosine as substrate in addition to its ability to convert phenylalanine to cinnamate. In such reactions the enzyme activity is designated tyrosine ammonia lyase (TAL). Conversion of tyrosine by TAL results in the direct formation of PHCA from tyrosine without the intermediacy of cinnamate.

Therefore, in one embodiment of the invention a host cell capable of producing CA and PHCA is used which comprises “PAL/TAL activity”, which refers to a protein which contains both PAL and TAL activity. Such a protein has at least some specificity for both tyrosine and phenylalanine as an enzymatic substrate.

However, all natural PAL/TAL enzymes prefer to use phenylalanine rather than tyrosine as their substrate. The level of TAL activity is always lower than PAL activity, but the magnitude of this difference varies over a wide range. Exception to this rule are the PAL/TAL enzymes from Rhodotorula spp. and from Rhodosporium toruloides (U.S. Pat. No. 4,636,466; Hanson and Havir in The Biochemistry of Plants; Academic: New York, 1981; Vol. 7, pp 577-625), in which a ratio of TAL catalytic activity to PAL catalytic activity is approximately 0.58. For that reason, a host cell is preferably provided with a PAL/TAL gene from Rhodotorula spp. or Rhodosporium toruloides. Alternatively, a host cell comprises a “modified PAL/TAL” activity which refers to a protein which has been derived from a wild type PAL enzyme but which has been genetically engineered such that the TAL activity is greater than PAL activity. As such, a modified PAL/TAL protein has a greater substrate specificity for tyrosine than for phenylalanine. Directions to obtain a modified PAL/TAL protein can be found in WO02/090523.

Unlike CA, PHCA can be completely degraded by the action of endogenous enzymes in Pseudomonads. This degradation route goes via PHB as an intermediate (Jimenez et al. 2002. Environ. Microbiol. 4:824-841). The present invention provides a method by which mutants are obtained that are no longer able to degrade PHCA or PHB

Thus, by introducing the TAL activity in a m-fluoro-phenylalanine resistant mutant of Pseudomonas, no longer able to degrade PHCA or PHB, efficient production of both aromatic acids is obtained.

In one embodiment, a host cell is genetically engineered with one or more foreign enzyme activities such that it produces an aromatic acid that is otherwise not produced in said host cell. In one embodiment, a host cell A is provided with the enzyme B from organism C to obtain a host cell comprising an efflux pump that is capable of producing aromatic acid D. Next to cinnamic acid and PHCA also benzoate and PHB can be aromatic acids which can be produced in host cells

Recombinant host cells can be obtained using methods known in the art for providing cells with recombinant nucleic acids. These include transformation, transconjugation, transfection or electroporation of a host cell with a suitable plasmid (also referred to as vector) comprising the nucleic acid construct of interest operationally coupled to a promoter sequence to drive expression. Typically, the plasmid also comprises a selection marker which confers the host cell with resistance to a selective agent, such as an antibiotic. Culturing the host cell in the presence of the selective agent, i.e. under a selective pressure, ensures that the plasmid is maintained by the host cell.

Thus, the present invention provides a method for providing a host that overproduces an aromatic acid, comprising introduction of PAL/TAL activity via introduction of a suitable plasmid and/or subjecting a population of host cells to random mutagenesis, followed by selection of host cells for increased m-fluoro-phenylalanine resistance, screening said resistant host cells for optimized aromatic acid production and selecting at least one mutated host that overproduces said aromatic acid compared to a parent host that has not been subjected to random mutagenesis.

However, selective agents are generally expensive. Especially in large scale microbial production systems, it is preferred to culture under the cheapest condition possible, i.e. without using selective pressure. In addition, it can be encountered that the host cell looses the extrachromosomal plasmid without loosing resistance against the selective agent. Therefore, a host cell for use in a method of the invention is preferably genetically modified using a procedure that does not rely on culturing under selection pressure and which results in a genetically stable recombinant host cell. In one embodiment, a host cell is provided with a nucleic acid of interest, for example a gene encoding an enzyme involved in aromatic acid synthesis such as PAL, using insertional mutagenesis. Herein, an isolated nucleic acid is inserted into the genome of the host cell. Insertion can be site-directed or random. In a preferred embodiment, the invention provides a method for providing a host cell (over)producing an aromatic acid comprising insertional mutagenesis. Insertional mutagenesis advantageously makes use of a transposon or a plasposon. A plasposon is a mini-transposon with an origin of replication (see Dennis and Zylstra, 1998). In a more preferred embodiment, random insertional mutagenesis is used to provide a genetically modified variant host cell for use in a method of the invention. For example, a collection of variant host cells, preferably P. putida, is provided which each contain a mini-transposon comprising the PAL gene randomly inserted into their genomic DNA. In each variant host cell, the genomic DNA will contain a mutation at the site of integration of the transposon. In a some cases, a mutation can lead to the inactivation of a genetic element (e.g. a coding region or a regulatory element) that is involved in aromatic acid metabolism in said host cell. For example, an enzyme may become inactivated which normally degrades or further metabolizes the desired aromatic acid produced by the host cell. This will of course contribute to an increased aromatic acid yield. Alternatively, a metabolic side-route is inactivated due to the insertional mutagenesis procedure such that the metabolic flux of precursor(s) into the biosynthesis of the aromatic acid is increased.

Thus, in one aspect, the invention provides a method for providing a host cell that overproduces an aromatic acid, comprising subjecting a population of host cells to random insertional mutagenesis, screening said mutated host cells (also referred to as ‘variant’ host cells) for optimized aromatic acid production and identifying at least one mutated host cell that overproduces said aromatic acid compared to a parent host cell that has not been subjected to random mutagenesis. Specifically such a method comprises selecting of a host cell for increased accumulation of phenylalanine and/or tyrosine by screening for mutants resistant against toxic analogs of an aromatic amino acid.

Variant host cells in which insertional mutagenesis has resulted in a useful mutant can be readily identified by screening for increased production levels of the desired compound. Since a host cell of the invention can secrete the aromatic acid into the medium, aliquots of the culture medium used for growing a variant host cell can be easily analyzed for the presence of increased aromatic acid production. Of particular interest are variant host cells wherein at least one enzyme involved in the degradation of said aromatic acid is disrupted or wherein a metabolic side-route of the biosynthesis of said aromatic acid is disrupted. Host cells according to the invention are cultured in an aqueous medium comprising a renewable fermentable carbon substrate according to standard microbial culturing conditions. The term “fermentable carbon substrate” refers to a carbon source capable of being metabolized by the host cell and particularly to carbon sources selected from the group consisting of monosaccharides (such as glucose), oligosaccharides, polysaccharides, polyols (such as glycerol) and one-carbon substrates or mixtures thereof. The preferred carbon substrates in a method of the invention for the production of an aromatic acid are glucose and glycerol.

Various culturing methods known in the art can be used, for example continuous, batch, semi-batch or fed-batch culturing. A person skilled in the art of biotechnology will be able to select which culturing method is most suitably used. In one embodiment, a method of the invention comprises culturing a host cell in a fed batch fermenter. In a fed batch fermenter the feed is continuously added until the maximum liquid fermenter volume is reached or until the cell population has reached its maximum density. The fermenter may be then allowed to continue or be partially or completely emptied, depending on the process.

Furthermore, the invention provides use of a host comprising an efflux pump for an aromatic acid, preferably a member of the proton-dependent resistance/nodulation/cell division (RND) family of efflux pumps, more preferably the solvent resistance pump srpABC of P. putida strain S12, for the microbial production of an aromatic acid. The host cell may be genetically modified to (over)produce the aromatic acid, for example by overexpression of at least one key enzyme in the aromatic acid biosynthesis

In a preferred embodiment, the host cell used is a Pseudomonas spp., preferably P. putida, more preferably P. putida strain S12. In another preferred embodiment, said host is used for the production of CA, para-hydroxycinnamic acid (PHCA) and PHB.

The invention is further illustrated by the examples below.

### LEGENDS TO THE FIGURE

FIG. 1. Schematic representation of the DNA constructs pTn-1 and pJWpalTn.

Abbreviations used: nagR/PnagAa, regulatory DNA sequence for naphthalene degradation from Comomonas testosteroni; rep, codes for the replication function; GmR, gentamycin resistance marker; bla, ampicillin resistance marker; Tn, transcription terminator; bp, basepairs

### EXAMPLES

### Example 1

### Cloning of a Nucleic Acid Sequence Encoding Phenylalanine Ammonia Lyase from R. toruloides into E. Coli-P. putida Expression Plasmids and Transformation into E. coli and P. putida S12

DNA encoding phenylalanine ammonia lyase (the pal gene; GenBank accession number X51513) was amplified using PCR from a cDNA collection obtained from R. toruloides mRNA as described by Sarkissian et al. (1999. Proc. Natl. Acad. Sci. USA 2:96) using oligonucleotides designed for the 5′- and 3′-end of the pal DNA. The oligonucleotide homologous to the 5′-end of pal (oligo 1) contains an additional KpnI restriction site and the oligonucleotide homologous to the 3′-end (oligo 2) an additional NotI restriction site (see Table 1).

TABLE 1

Nucleic acid sequence of the oligonucleotide
used for amplification of de DNA's described
in Examples 1 to 4.

| | | DNA to |
|---|---|---|
| Oligonu- | | scale |
| cleotide | Sequence (5′→3′) | up |
| 1 | gcggtaccatggcaccctcgctcgactcgatc | pal |
| 2 | gcgcggccgcctaagcgagcatcttgaggagg | pal |
| 3 | gcactagtgcacaagaccagtcgcatgggagaac | nagR/ |
| | | PnagAa |
| 4 | ctggtgagacatgggaagcggcc | nagR/ |
| | | PnagAa |

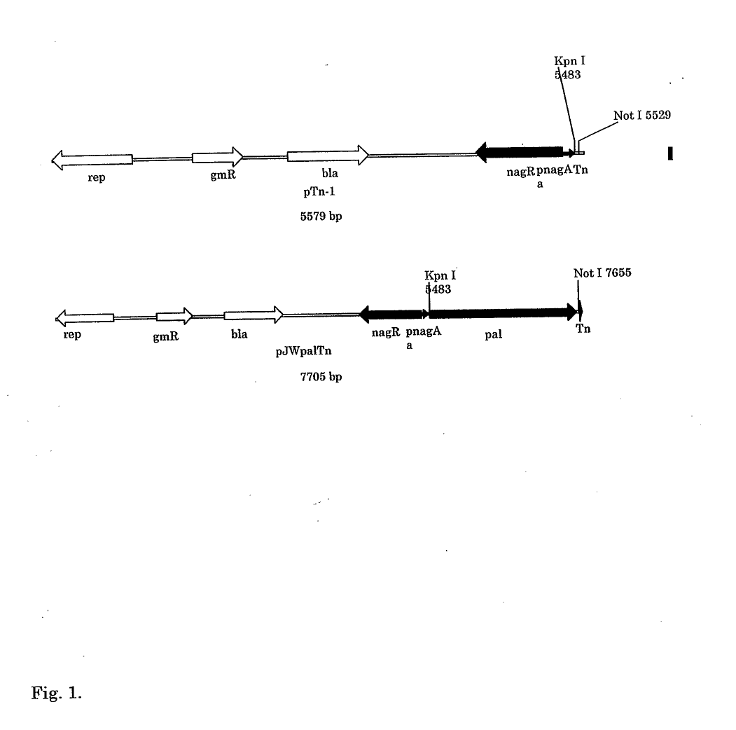

The PCR-amplified pal gene was digested with KpnI and NotI and ligated into the plasmid pTn-1 (FIG. 1.) which had also been digested with KpnI and NotI. This resulted in the DNA construct pJWpalTn (FIG. 1) wherein the pal gene was placed under the control of the inducible regulatory sequence nagR/PnagAa (Hüsken et al. 2001. Appl. Microbiol. Biotechnol. 55:571-577). Hereinafter, this configuration is referred to as the nagR/PnagAa::pal cassette.

PJWpalTn was introduced into E. coli and P. putida S12 host cells by standard transformation procedures. P. putida S12 genetically engineered to express phenylalanine ammonia lyase is referred to as S12pal. The host cells were cultured under selection pressure (ampicillin for E. coli and gentamycin for P. putida S12) to ensure that the host cells maintained the plasmid.

### Example 2

### Generation of a Collection of P. putida S12pal Mutants Resistant to M-Fluorophenylalanine and Screening for Mutants with Increased CA and PHCA

The availability of phenylalanine and tyrosine in S12pal was optimized in order to improve production of CA and PHCA. It is known from the art that toxic phenylalanine analogs (e.g. m-fluoro-phenylalanine) can be used to select for mutants that overproduce phenylalanine and/or tyrosine. This approach was used in the present invention to obtain a production strain optimized for the production of CA and PHCA. S12pal was mutagenized by treatment with the mutagenic agent NTG to obtain a bank of 20.000 m-fluorophenylalanine resistant mutants.

The mutants in this bank were subsequently tested for their ability to overproduce CA and PHCA in comparison to parent strain S12pal. The absorbance of light at a wavelength of 278 nm and 310 nm of the culture medium of all mutants was determined as a relative measure for CA and PHCA, respectively. Different mutants were selected which exhibited an increased production of either CA or PHCA. A CA overproducing mutant, referred to as S12pal1, and a PHCA overproducing mutant, referred to as S12pal2, were tested in more detail for the production of respectively CA and PHCA from the renewable carbon substrate glucose during growth (Example 4).

### Example 3

### Selection of Mutants with Increased PHB Production from a Bank of m-fluorophenylalanine Resistant S12pal Mutants

It is established in the art that P. putida is able to convert PHCA into PHB. PHB can subsequently be degraded by these bacteria. This is exemplified by the fact that P. putida S12 is able to grow on PHB as the sole carbon source. In order to select for S12pal mutants that show an increased production of PHB, the bank of m-fluorophenylalanine resistant S12pal mutants obtained in Example 2 was first screened for mutants that were no longer able to utilize PHB for growth. To this end individual mutants of the bank were transferred into an aqueous mineral medium (Hartmans et al. 1989. Appl. Environ. Microbiol. 55:2850-2855) supplemented with 20 mM of PHB as the sole carbon source. Different mutants were obtained no longer able to grow on PHB. One such mutant, S12pal3, was tested in more detail for the production of PHB from glucose (Example 4).

### Example 4

### Production of CA, PHCA and PHB by Batch-Wise Cultivated S12pal, S12pal1, S12pal2 and S12pal3

To determine the production of CA, PHCA and PHB by, respectively, S12pal1, S12pal2, S12pal3 and their parent S12pal, these strains were cultured batch-wise in an aqueous mineral medium (Hartmans et al. 1989. Appl. Environ. Microbiol. 55:2850-2855). This medium was supplemented with 20 mM glucose, and 10 mg/L gentamicin to ensure plasmid maintenance. In order to induce expression of the pal gene into a functional phenylalanine ammonia lyase, 0.1 mM sodium salicylate was also supplemented. As a control for the source of phenylalanine ammonia lyase activity in S12pal, we also tested a P. putida variant provided with the pTn-1 plasmid without the pal gene. All strains were cultivated in the presence or absence of 1 mM exogenously added phenylalanine or tyrosine. The levels of CA, PHCA and PHB, in the culture supernatants were determined at various time points using High Performance Liquid Chromatography (HPLC). In addition cell growth was monitored by determining total cellular protein content in the cultures at various time points.

In addition, at various time points cell free extracts from S12pal were prepared from the cultures which were analysed for protein content and phenylalanine ammonia lyase activity. Hereto, the formation of CA and PHCA was determined using HPLC at various time points following the addition of 1 mM phenylalanine or 1 mM tyrosine to the cell free extracts.

The results of these measurements were used to determine the following parameters for CA, PHCA and PHB production (see Table 2):
the maximal concentration in the culture medium or the cell free extract

the maximal specific rate of production in the culture medium or the cell free extract.

TABLE 2
Production of CA, PHCA and PHB in batch cultures and cell-free
extracts of mutants of the P. putida S12, genetically modified
with the gene encoding phenylalanine ammonia lyase (PAL).

| | Maximum | | | Maximum specific | | |
|---|---|---|---|---|---|---|
| | concentration^{b)} | | | production rate^{c)} | | |
| | (μM) | | | (μmol · min⁻¹ · g⁻¹) | | |
| Cultures or CVE^{a)} | CA | PHCA | PHB | CA | PHCA | PHB |
| S12 control | 0 | 0 | 0 | 0 | 0 | 0 |
| S12pal | 72 | 7 | 0 | 0.3 | 0.2 | 0 |
| S12pal1 | 415 | Nd | 0 | 2.3 | Nd | 0 |
| S12pal2 | 278 | 116 | 0 | 1.0 | 1.1 | 3.2 |
| S12pal3 | 150 | 0 | 178 | 0.9 | 0 | 0.7 |
| S12 control + fen. | 0 | 0 | Nd | 0 | 0 | Nd |
| S12pal + fen. | 227 | 29 | Nd | 1.1 | 1.1 | Nd |
| S12pal1 + fen. | 627 | Nd | Nd | 3.2 | Nd | Nd |
| S12 control + tyr. | 0 | 0 | Nd | 0 | 0 | Nd |
| S12pal + tyr. | 149 | 207 | Nd | 0.8 | 2.5 | Nd |
| S12pal1 + tyr. | 102 | 26 | Nd | 1.1 | 1.1 | Nd |
| CVE S12 control + fen. | 0 | 0 | Nd | 0 | 0 | Nd |
| CVE S12pal + fen. | nd | nd | Nd | 39 | nd | Nd |
| CVE S12 control + tyr. | 0 | 0 | Nd | 0 | 0 | Nd |
| CVE S12pal + tyr. | nd | nd | Nd | nd | 3.8 | Nd |

^{a)}CVE, cell free extract; S12 control, P. putida S12 with vector pTn-1; S12pal, P. putida S12 with construct pJWpalTn; S12pal1, a m-fluorophenylalanine resistant mutant derived from S12pal selected for CA overproduction; S12pal2, a m-fluorophenylalanine resistant mutant selected for PHCA overproduction, S12pal3, a m-fluorophenylalanine resistant mutant selected for overproduction of PHB, + fen., with 1 mM phenylalanine added; + tyr, with 1 mM tyrosine added.
^{b)}The maximal concentration CA en para-hydroxycinnamic acid in the culture medium or CVE as determined by HPLC.
^{c)}The maximum specific production rate is defined as the maximum amount of CA, PHCA or PHB (in micromol) that accumulates per minute per gram cell protein in the culture medium or CVE. Nd = not determined.

1. A method for the enzymatic production of an aromatic acid in a microbial host cell from a renewable carbon substrate, wherein said host cell comprises an efflux pump for said aromatic acid.

2. A method according to claim 1, wherein said efflux pump is a member of the proton-dependent resistance/nodulation/cell division (RND) family of efflux pumps, preferably a solvent resistance pump, more preferably the solvent resistance pump srpABC of P. putida strain S12.

3. A method according to claim 1, wherein said host cell is a Pseudomonas spp., preferably P. putida, more preferably P. putida strain S12.

4. A method according to claim 1, wherein said host cell expresses or overexpresses at least one enzyme involved in the biosynthesis of said aromatic acid.

5. A method according to claim 1, wherein said host cell is genetically modified to produce or overproduce said aromatic acid or a precursor thereof.

6. A method according to claim 1, herein said aromatic acid is selected from the group consisting of cinnamic acid, parahydroxycinnamic acid and para-hydroxybenzoic acid.

7. A method according to claim 6, wherein said host cell overexpresses phenylalanine ammonia lyase (PAL), preferably PAL with tyrosine ammonia lyase (TAL) activity.

8. A method according to claim 1, wherein said host cell is selected for increased accumulation of phenylalanine and/or tyrosine by screening for mutants resistant against toxic analogs of an aromatic amino acid.

9. A method according to claim 1, wherein said renewable carbon substrate is selected from the group consisting of monosaccharides, oligosaccharides, polysaccharides, polyols, methanol, formaldehyde, formate, and carbon-containing amines, preferably glucose and glycerol.

10. A method for providing a host cell that overproduces an aromatic acid, comprising introducing PAL/TAL activity in a population of host cells, subjecting said population to random mutagenesis, selecting mutant host cells for increased m-fluoro-phenylalanine resistance, screening said selected mutant host cells for increased aromatic acid production and selecting at least one mutated host cell that overproduces said aromatic acid compared to a parent host cell that has not been subjected to random mutagenesis.

11. A host cell obtainable by a method according to claim 10.

12. A host cell according to claim 11, wherein at least one enzyme involved in the degradation of said aromatic acid is disabled or wherein a metabolic side-route of the biosynthesis of said aromatic acid is disabled.

13. Use of a host cell comprising an efflux pump, preferably a member of the proton-dependent resistance/nodulation/cell division (RND) family of efflux pumps, more preferably the solvent resistance pump srpABC of P. putida strain S12, for the microbial production of an aromatic acid from a renewable carbon substrate.

14. Use according to claim 13, wherein said host cell is a Pseudomonas spp., preferably P. putida, more preferably P. putida strain S12.

15. Use according to claim 13, wherein said aromatic acid is cinnamic acid, para-hydroxycinnamic acid and para-hydroxybenzoic acid.

16. A method according to claim 2, wherein:
said host cell is a Pseudomonas spp., preferably P. putida, more preferably P. putida strain S12;
said host cell expresses or overexpresses at least one enzyme involved in the biosynthesis of said aromatic acid;
said host cell is genetically modified to produce or overproduce said aromatic acid or a precursor thereof;
said aromatic acid is selected from the group consisting of cinnamic acid, parahydroxycinnamic acid and para-hydroxybenzoic acid;
said host cell overexpresses phenylalanine ammonia lyase (PAL), preferably PAL with tyrosine ammonia lyase (TAL) activity;
said host cell is selected for increased accumulation of phenylalanine and/or tyrosine by screening for mutants resistant against toxic analogs of an aromatic amino acid; and
said renewable carbon substrate is selected from the group consisting of monosaccharides, oligosaccharides, polysaccharides, polyols, methanol, formaldehyde, formate, and carbon-containing amines, preferably glucose and glycerol.

17. Use according to claim 14, wherein said aromatic acid is cinnamic acid, para-hydroxycinnamic acid and para-hydroxybenzoic acid.
